(19) United States
(12) Patent Application Publication
(10) Pub. No.: US 2008/0242955 A1
(43) Pub. Date: Oct. 2, 2008

(54) RELIABILITY IN DETERMINATION OF CLINICAL STATE OF A SUBJECT

(76) Inventors: Kimmo Uutela, Helsinki (FI); Borje Rantala, Helsinki (FI); Minna Kymalainen, Helsinki (FI); Matti Huiku, Espoo (FI); Petri Karkas, Espoo (FI)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/693,895

(22) Filed: Mar. 30, 2007

(51) Int. Cl. A61B 5/00

(52) U.S. Cl. 600/301

(57) ABSTRACT

The invention relates to the determination of a diagnostic index indicative of the clinical state, especially nociceptive state, of a subject. In order to improve the quality of the determination process, desired input signals derived from the subject are monitored and the quality of the process is improved based on the monitoring. This is implemented by increasing user awareness of the reliability of the index or by controlling the measurement set-up. Quality information indicative of the reliability of the current index values may be produced based on the input signals. The quality information may then be employed to give an indication of the current reliability of the index and a warning if the reliability of the diagnostic index becomes compromised.

### FIELD OF THE INVENTION

The present invention relates generally to improvement of reliability in connection with the determination of a clinical state of a subject, such as the nociceptive or antinociceptive state of a patient. The clinical state here refers to a physiological status of the subject, which is indicative of a need or effect of a treatment or intervention, where the term physiological relates to physiology, the science dealing with the functions of living body and beings.

### BACKGROUND OF THE INVENTION

During the past few years, several commercial devices for measuring the level of consciousness and/or awareness in a clinical set-up during anesthesia have become commercially available. These devices, which are based on a processed one-channel EEG signal, have been introduced by Aspect Medical (Bispectral Index), by Datex-Ohmeda (Entropy Index) and by Danmeter (an auditory evoked EEG potential monitoring device, AAI™). At present, the situation with the assessment of the cortical activity and integrity is considered satisfactory, though not resolved for all applications.

As to the central nervous system (CNS), the assessment or measurement of the suppression of the sub-cortical activity, the autonomic nervous system (ANS) and the integrity of subcortical evaluations is far more unsatisfactory. No commercial devices exist for this purpose. This is mainly because the sub-cortical components are not represented in any single bioelectrical or other signal, in contrast to the fact that the EEG almost alone may represent the cortical activity. In addition to the monitoring of the hypnotic state of the brains by EEG, the monitoring of the adequacy of anesthesia or sedation thus calls for a multi-parameter approach that combines parameters describing the overall responsiveness of the patient to “unconscious” stimulations.

The sub-cortical integrity of the afferent input, ANS evaluations, and efferent autonomic output is best researched in unconscious subjects with noxious stimulations and their responses, as these are mainly processed and modulated in the brainstem and spinal levels. The responses can also be modulated (attenuated) by analgesics or antinociceptive drugs, which influence the pain pathways at the sub-cortical levels. A successful monitoring method shall thus demonstrate a clear relationship and correlation between both the effect of the analgesics on the suppression of the nociceptive responses and the intensity of the noxious stimulations on the strength or amount of the responses in the parameters.

The need for reliable monitoring of the adequacy of anesthesia is based on the quality of patient care and on economy related aspects. Balanced anesthesia reduces surgical stress and there is firm evidence that adequate analgesia decreases postoperative morbidity. Awareness during surgery with insufficient analgesia may lead to a post-traumatic stress disorder. Prolonged surgical stress sensitizes the central pain pathways, which post-operatively increases patient pain and secretion of stress hormones. Low quality pre- and intra-operative analgesia makes it difficult to select the optimal pain management strategy later on. More specifically, it may cause exposure to unwanted side effects during the recovery from the surgery. Too light an anesthesia with insufficient hypnosis causes traumatic experiences both for the patient and for the anesthesia personnel. From economical point of view, too deep an anesthesia may cause increased perioperative costs through extra use of drugs and time, and also extended time required for post-operative care. Too deep a sedation may also cause complications and prolong the usage time of expensive facilities, such as the intensive care theater.

The sympathetical nervous system usually prepares us for high stress situations by speeding up the body functions. Under conditions of normal ANS regulation, the parasympathetical system restores the normal conditions in blood circulation by slowing down the heart rate. Pain, discomfort, and surgical stress may activate the sympathetical branch of the ANS and cause an increase in blood pressure, heart rate and adrenal secretions. Therefore, the automatic methods that have been suggested for assessing stress and pain during anesthesia or sedation rest on the fact that during periods of stress or pain, the activity of the sympathetic nervous system increases, while the activity of the parasympathetic nervous system decreases.

WO 03/084396 discloses a system and method of assessment of pain and stress during anesthesia or sedation, which employs Pulse Wave Velocity (PWV) and Pulse Transit Time (PTT) to obtain an indication of sympathetic activity. PWV is the velocity of the pressure wavefront propagating along an arterial tree. PTT is the time for the wave front to travel a fixed distance. During periods of increased sympathetic activity PWV increases and PTT decreases.

One drawback of this system is that it requires continuous availability of both ECG and photoplethysmographic (PPG) waveforms.

A wider selection of physiological signals is available for the assessment of pain and stress in a system disclosed in U.S. Patent Application 2006/0217614. In this system, a measure of the clinical state of a patient may be generated directly based on a physiological signal indicative of the function of the cardiovascular system by applying to said signal a normalization transform dependent on predetermined history data, such as previously measured values of the same signal. The system enables the monitoring of the trend of the clinical state, such as the level of nociception, and also the verification of the current clinical state against a fixed scale. To improve the specificity of the said measure, a composite indication may be produced based on a plurality of signals or parameters obtained from the patient. In a typical application, the nociceptive state of a patient is determined by calculating an index of nociception as a weighted average of two or more normalized parameters derived from the physiological signals measured from the patient. If two parameters are employed, the index may be produced by calculating a weighted average of a first parameter indicative the amplitude of a first physiological signal and a second parameter indicative of the pulse interval of the patient. The first physiological signal may be, for example, a plethysmographic signal. In a simple implementation, the nociceptive index may thus be produced by combining plethysmographic amplitude information and heart rate information.

The above automatic mechanisms for measuring an index indicative of the pain, discomfort, or surgical stress thus rest on the physiological feature that pain, discomfort and surgical stress activate the sympathetical branch of the autonomic nervous system. Although the above-mentioned system allows a wide selection of physiological signals to be used for the assessment of pain or stress, the efforts for accomplishing a measure that may be produced in different clinical environments regardless of the physiological signals available in each case are complicated by the fact that no definite parameters exist for describing pain or stress. Since the ability of different physiological signals or parameters to reflect pain or stress may vary, the functionality of a measurement system may also vary depending on the patient and on the combination of physiological signals and parameters available for the measurement. Therefore, the reliability of the measurement system may also vary without the user being aware of it.

The present invention seeks to alleviate or eliminate this drawback.

### SUMMARY OF THE INVENTION

The present invention seeks to provide a novel mechanism for improving the quality of the assessment of the clinical state, especially the level of pain and stress, of a subject. The present invention further seeks to accomplish a mechanism that may be used to enhance the quality of the measurement by enhancing user awareness of the current reliability of the measurement.

In the present invention, the set of physiological signals available from the subject and/or parameters derived from the physiological signals are monitored and the quality of the index determination process is enhanced based on the monitoring results by enhancing the quality of the information provided to the user. Based on the monitoring process, an indication may be given to the user of the apparatus to give him/her a notion of the current level of reliability of the diagnostic index. Alternatively, the results of the monitoring process may be used to control the measurement set-up, thereby to enhance the reliability of the index, with or without a separate reliability indication to the user. In other words, the invention improves the quality of the overall index determination process, which determines the index and indicates it to the user, by increasing user awareness of the current reliability of the index or by changing the measurement set-up so that the reliability of the index is increased.

Thus one aspect of the invention is providing a method for ascertaining the clinical state of a subject. The method includes acquiring at least one physiological signal from a subject, deriving at least one parameter sequence from at least one desired physiological signal belonging to the at least one physiological signal, and performing an index determination process, thereby to form a diagnostic index based on the at least one parameter sequence, wherein the diagnostic index serves as a measure of the clinical state of the subject and wherein the index determination process includes indicating the diagnostic index to a user. The method further includes monitoring at least one input signal, wherein the at least one input signal belongs to a group including the at least one physiological signal and the at least one parameter sequence and enhancing, based on the monitoring, the quality of the index determination process.

Although the number of physiological signals available and the number of parameter sequences derived from each physiological signal may vary, in a typical application, in which the nociceptive state of a patient is determined, an ECG signal and a plethysmographic signal are employed to determine the index of nociception. If other physiological signals are not available from the patient, pulse transit time (PTT) data derived from the said signals or heart rate (HR) data may be used to produce a signal indicative of the reliability of the index of nociception. Alternatively, if a blood pressure signal is available from the patient, the said signal may be used to detect quality variations in the index.

Another aspect of the invention is that of providing an apparatus for ascertaining the clinical state of a subject. The apparatus includes a measurement unit configured to acquire at least one physiological signal from a subject, a first calculation unit configured to derive at least one parameter sequence from at least one desired physiological signal belonging to the at least one physiological signal, and a second calculation unit configured to perform an index determination process, thereby to form a diagnostic index based on the at least one parameter sequence, wherein the diagnostic index serves as a measure of the clinical state of the subject and wherein the index determination process is configured to indicate the diagnostic index to a user. The apparatus further includes a quality enhancing module configured to monitor at least one input signal and to enhance the quality of the index determination process based on the at least one input signal, wherein the at least one input signal belongs to a group including the at least one physiological signal and the at least one parameter sequence.

In one embodiment of the invention, a quality measure is produced, which indicates the current reliability of the index. The index may be displayed as a graph to the user of the apparatus.

In a further embodiment of the invention, the physiological signals available from the subject may be monitored continuously in order to figure out whether the selection of signals used for the determination of the index needs to be changed due to the reason that the reliability of the index obtained through the current signal selection is degraded or begins to degrade.

A further aspect of the invention is that of providing a computer program product by means of which a known measurement device may be upgraded to improve the quality of the measurement of the clinical state of the patient. The program product includes a first program code portion configured to monitor at least one input signal belonging to a group including at least one physiological signal acquired from a subject and at least one parameter sequence derived from at least one desired physiological signal belonging to the at least one physiological signal and a second program code portion responsive to the first program code portion and configured to enhance the quality of an index determination process configured to determine a diagnostic index based on the at least one parameter sequence, wherein the diagnostic index serves as a measure of the clinical state of the subject.

Other features and advantages of the invention will become apparent by reference to the following detailed description and accompanying drawings.

### BRIEF DESCRIPTION OF THE DRAWINGS

The drawings illustrate the best mode presently contemplated of carrying out the invention. In the following, the invention and its preferred embodiments are described more closely with reference to the examples shown in FIG. 1a to 10 in the appended drawings, wherein:

FIG. 1a illustrates the main processes and the corresponding signals and parameters in the measurement set-up of the invention;

FIG. 1b is a flow diagram illustrating one embodiment of the invention;

FIG. 2 is a flow diagram illustrating a further embodiment of the invention;

FIG. 3 illustrates one embodiment of the determination of the index of nociception in the embodiment of FIG. 2;

FIG. 4 illustrates an alternative for the embodiment of FIG. 2;

FIG. 5 illustrates the enhancement of the index quality in the embodiments of FIGS. 2 and 4;

6a to 6d illustrate the detection of an error situation in the index determination;

FIG. 7 illustrates a further embodiment of the invention for enhancing the quality of the measurement of the diagnostic index;

FIG. 8 illustrates one embodiment of a system according to the invention;

FIG. 9 illustrates the operational units of the control unit of FIG. 8; and

FIG. 10 illustrates an example of the presentation of the diagnostic index and the reliability thereof to the user of the apparatus.

### DETAILED DESCRIPTION OF THE INVENTION

As discussed above, automatic methods have been suggested for defining an index indicative of stress and pain during anesthesia or sedation. The index may be a composite measure determined based on a plurality of physiological signals or parameters obtained from a subject. For example, the measure may be derived from ECG and plethysmographic (PG) signals obtained from the subject. In this context, the abbreviation PG covers both plethysmographic and photoplethysmographic signals/parameters.

However, some of the signals or parameters employed for producing the said measure may not always be direct measures of a stress response. For example, heart rate (HR) and especially plethysmographic amplitude (PGA) may be changed by different kinds of medication, not just pain medication. Some conditions, such as the temperature of the patient, may also affect the PGA. Furthermore, PGA is not directly linked to vasoconstriction; changes in the operation of the heart and changes in the amount of fluid or fluid balance may affect the PGA. Therefore, the user of the apparatus of the invention may be provided with information indicating when the functionality of the index determination process becomes challenged or compromised due to a reason that one or more of the physiological signals or parameters used to define the diagnostic index change due to reasons not directly related to stress or analgesia.

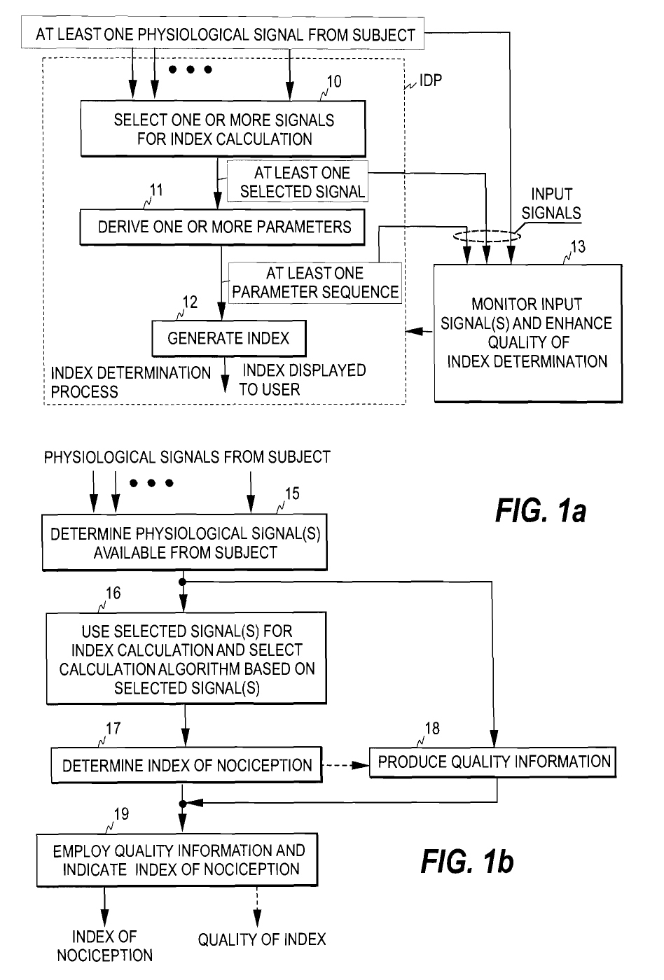

FIG. 1a illustrates the main process steps and the corresponding signals and parameters in the measurement set-up of the invention. In the measurement set-up, at least one but typically several physiological signals are acquired from a subject. From among the acquired physiological signals one or more desired signals are selected at step 10 to be used for the index determination. From the said at least one desired physiological signal at least one parameter sequence is then generated at step 11, whereby at least one parameter sequence is obtained. It is to be noted here that in the index determination process IDP one or more time series are derived from the desired physiological signal(s). The time series derived from the original physiological signals are in this context termed parameters or parameter sequences to make a distinction between the original physiological signal(s) and the time series of the parameters derived from the physiological signal(s). It is further to be noted here that the number of parameter sequences is not necessarily equal to the number of the selected physiological signals, since more than one parameter may be calculated from a single physiological signal selected. For example, if an ECG signal is selected, both heart rate (HR) and heart rate variability (HRV) may be derived from the ECG signal. The parameter(s) are then employed to determine the index (step 12) and from among the physiological signals and the parameter sequences available in the measurement set-up at least one signal/parameter that is relevant with respect to the reliability of the index determination is used as an input signal for a monitoring process (step 13) which enhances, based on the input signal(s), the quality of the index determination process IDP (step 10-12) by enhancing the quality of the information provided to the user.

FIG. 1b is a flow diagram illustrating one embodiment of the invention for determining of an index of nociception. The physiological signal(s) available from the subject are first determined (step 15) and at least one of them is selected for the determination of the index of nociception. Although this selection may be automatic, it may also be performed by the user of the apparatus. Based on the signal(s) selected, an algorithm is selected for calculating the index of nociception (steps 15 and 16). For example, if the physiological signals available from the subject include an ECG signal and a plethysmographic signal, they may be selected for determining the index of nociception. The selection of the algorithm here refers to the determination of an algorithm instance specific to the physiological signal or signal combination selected for the determination the index.

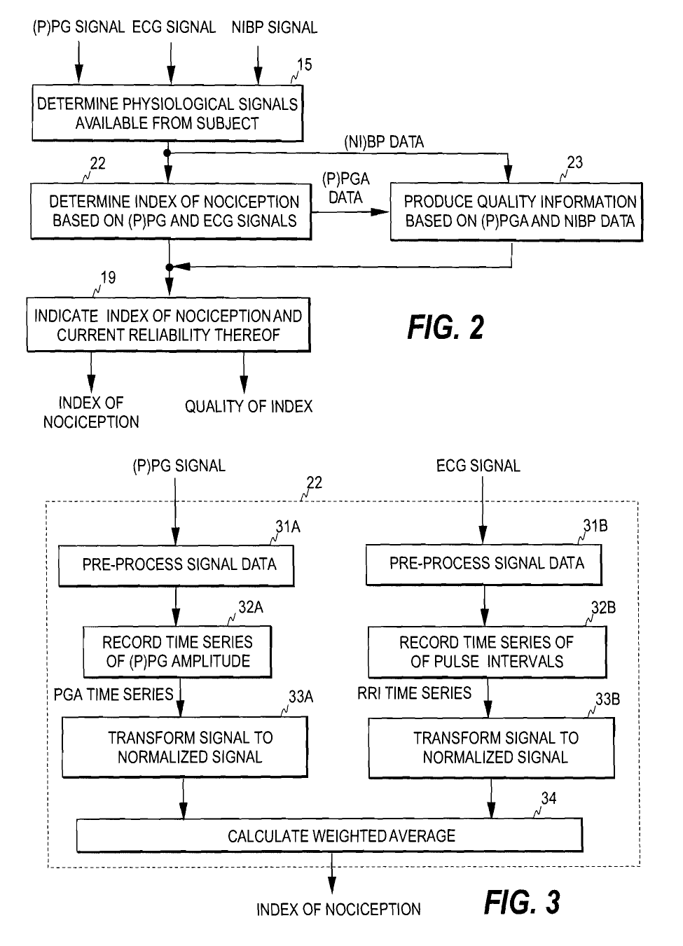

The algorithm is then employed to determine the index of nociception (step 17). This may be carried out as disclosed in the above-mentioned U.S. Patent Application 2006/0217614, for example. As discussed therein, at least a physiological signal indicative of the function of the cardiovascular system of the subject is used for determining the index. Such a signal may be a plethysmographic signal (PG), such as a photoplethysmographic (PPG) signal, a blood pressure (BP) signal, an ECG signal, or a Laser Doppler flow signal in peripheral tissues. Typically, however, the index is calculated based on a plethysmographic signal and an ECG signal, as is discussed below in connection with FIG. 2.

With reference to FIG. 1b, the physiological signals available from the subject are utilized to produce quality information indicative of the quality of the diagnostic index produced (step 18). Since the determination of the index of nociception at step 17 may involve utilization of the same physiological signal(s) as the production of the quality information at step 18, a parameter sequence required for the production of the quality information may be available from step 17, cf. dashed arrow in the figure, whereby the computation load of step 18 may be reduced.

The quality information may be produced solely on the basis of one or more of the physiological signals available or solely on the basis of the parameter sequences employed for the index determination. For example, a quality level may be determined based on the particular physiological signals available for the index determination. Generally, if only one physiological signal is available for the determination of the diagnostic index, the quality is lower than if one or more additional physiological signals are available (provided that the additional signals have relevant information content). Different physiological signals and their combinations may be mapped to respective reliability levels and the level may be indicated according to the physiological signal(s) used at each time.

In further embodiments of the invention, one or more time series which are not used for the determination of the index may be employed to indicate if the validity of the algorithm used to calculate the diagnostic index will be challenged. As is discussed below in connection with FIGS. 2 and 5, these time series may be derived from physiological signals which are or are not used for the determination of the index.

The diagnostic index calculated in step 17 is displayed to the user and the quality information available from step 18 is employed to improve the quality of the index measurement (step 19). In some embodiments of the invention, the quality information is utilized to indicate the reliability of the index to the user. In these embodiments, different quality/reliability levels may be indicated by different visual cues, such as colors, bars or fonts. However, it is also possible to calculate a quality measure and indicate its current value to the user of the apparatus. In some other embodiments of the invention, the quality information may be utilized to automatically enhance the quality of the measurement, with or without a reliability indication to the user.

In a typical application, the diagnostic index is determined based on the time series of two parameters. Although the two time series may be derived from one physiological signal, it is preferable to use two physiological signals of different types. The first time series typically represents the pulse amplitude of a plethysmographic waveform signal, while the second time series represents the beat-to-beat interval, i.e. pulse interval, of a plethysmographic signal, an ECG signal, or a blood pressure signal.

FIG. 2 illustrates an embodiment in which the physiological signals obtained from the subject include a PG signal, (NI)BP ((non-invasive) blood pressure) signal, and an ECG signal. Since PG and ECG signals are available in this case, the index of nociception is determined based on both signals at step 22. In this example, one parameter sequence (time series) is derived from the PG signal, while another parameter sequence (time series) is derived from the ECG signal.

FIG. 3 illustrates an example of the determination of the index of nociception at step 22 of FIG. 2. The recorded waveform data may first be pre-processed at steps 3 1A and 3 1B for filtering out some of the frequency components of the signal or for rejecting artifacts, for example. These steps are not necessary, but may be performed to improve the quality of the signal data. Next, the pulse amplitude of the PG signal is extracted at step 32A, whereby a first time series representing PGA is obtained. Simultaneously, a second time series representing the pulse interval (beat-to-beat interval, RRI) is produced at step 32B.

The two time series are then subjected to respective normalization processes in steps 33A and 33B. The normalization process here refers to a process that scales the input signal values to a predetermined output value range, such as 0 to 100. The normalized PG amplitude and the normalized pulse interval are then combined at step 34 to form a composite indicator that serves as the index of nociception. The composite indicator may be calculated as the weighted average of the two signals, in which the weights are specific to the PGA/RRI combination employed to determine the index of nociception.

Since the core of the present invention does not relate to the determination of the index and since the determination may be carried out in various ways, it is not described in more detail in this context. The above-mentioned U.S. Patent Application 2006/0217614, the content of which is incorporated by reference herein, discloses various methods for the index determination.

With reference to FIG. 2 again, the PGA time series obtained in step 22 and the (NI)BP ((non-invasive) blood pressure) signal data obtained from the subject may be employed in step 23 to produce quality information that indicates error situations.

Generally, the determination of the index of nociception assumes that with higher stress heart rate and vasoconstriction increase and that blood pressure (BP) and PGA are related to each other and to vasoconstriction in a particular way. More specifically, the PPGA signal is indicative of variation in local blood volume. These volume changes can be expressed as follows: PGA=D×BPPA, where D represents the distensibility of the vascular wall and BPPA represents the blood pulsed pressure amplitude, i.e. the difference of the systolic and diastolic pressures. Nociception and surgical stress causes a sympathetic activation that increases blood pressure and decreases D through vasoconstriction. The change in the PGA (APGA) may be expressed as follows: ΔPGA=ΔD×BPPA+D×ΔBPPA. During increased stress vasoconstriction is normally the dominating factor (i.e. the first term of the equation). Vasoconstriction normally increases BP and also BPPA, since the “resistance” of peripheral blood circulation increases. When blood pressure and PGA react consistently to stress, PGA decreases while BP and BPPA increase. However, if the patient suffers from stenosis or if the distensibility of the arteries is lowered, inconsistent behavior may be detected, i.e. PGA increases when BP increases. This kind of change may be comprehended by the above equation so that when the wall of the blood vessel is inelastic (thereby, ΔD=0), a change in BPPA directly causes a change in PGA. A similar situation may occur when an external factor, such as hypothermia, has already caused almost a maximum constriction of blood vessels. Measuring BP and PGA simultaneously, an inconsistent situation may be detected.

Another parameter indicative of a paradoxical change in a PG signal is the pulse transit time (PTT). In this context, PTT can be defined, for instance, as the time interval between an R peak in the ECG and the maximum slope or the time of reaching the half height of the following PGA pulse. Changes in PTT are usually related to the changes of pressure wave velocity. This is mainly determined by the distensibility D of the blood vessel (wavefront velocity of a pressure wave in a fluid filled tube depends mainly on the properties of tube walls, not on the pressure level). It is further to be noted that in this case the whole path from the heart to the site of measurement is important, i.e. in this case D characterizes the distensibility of the whole path, while the distensibility D in case of PGA changes is related to the local distensibility at the measurement site. Although PTT may not be robust enough for measuring the index of nociception, changes in PGA and PTT are consistent under normal conditions, i.e. vasoconstriction decreases D and consequently both PTT and PGA decrease. A paradoxical change in PGA can thus be detected using PTT as a comparison.

Thus, the quality of the measurement of the index of nociception may be assessed by observing the changes occurring simultaneously in PGA and in another parameter/signal, which may be PTT or BP. As discussed below, PGA variability or heart rate variability (HRV) may also be employed as said parameter.

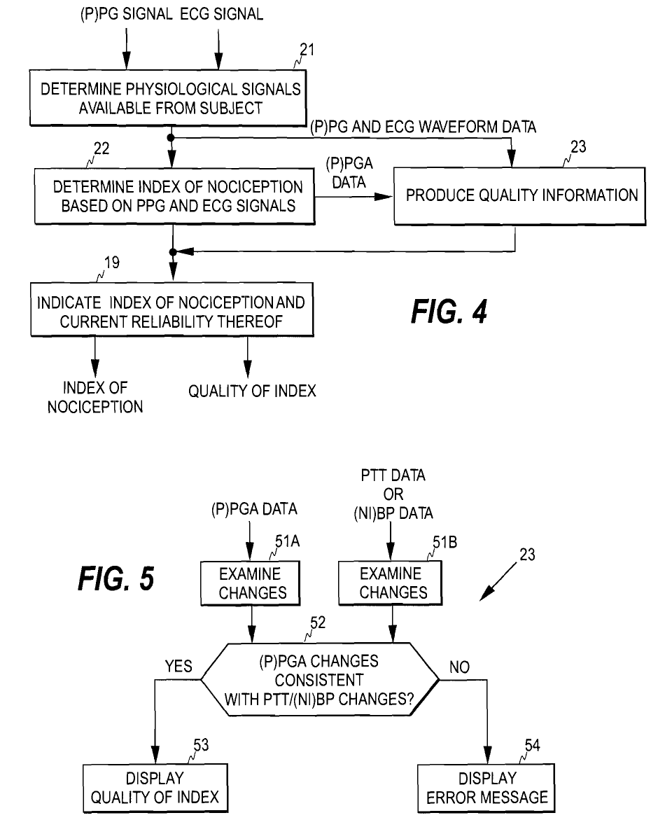

In the embodiment illustrated in FIG. 2, the quality information is thus produced based on (NI)BP and PGA data in step 23. This embodiment thus requires that a (NI)BP signal is available from the subject. In the embodiment of FIG. 4, the quality information is produced by comparing changes in PGA with changes in PTT. The PTT values may be calculated in step 23 based on the ECG and PG data by identifying the R peaks from the ECG waveform and the corresponding points of reaching half pulse heights from the PG waveform.

FIG. 5 illustrates one embodiment of the operations carried out in step 23 of FIGS. 2 and 4. The waveforms or time series of PGA and PTT/(NI)BP are examined in steps 51A and 51B, respectively, to see how the said parameters change over time. As discussed above, in the embodiment of FIG. 2, (NI)BP data is input to step 51B, while in the embodiment of FIG. 4 PTT data is input to step 5IB. The changes detected are compared with each other in step 52. If an inconsistency is detected, an error message is displayed to warn the user of the apparatus that the index readings may be erroneous (step 54). If PGA changes in a consistent manner with changes detected in PTT/(NI)BP, a high quality of the index may be indicated and displayed to user(step 53).

In one embodiment of the invention, steps 51 and 52 may be carried out by calculating the correlation of log(P)PGA and PTT within a sliding time window having a predetermined length, such as one minute. If this quality measure drops below a predetermined threshold, error is detected and the process proceeds to step 54 to display a warning message. If the correlation remains above the threshold, the apparatus may just indicate the value of the quality measure and/or use visual cues which indicate the quality level corresponding to the current quality measure. Correlation between (NI)BP and PGA or between heart rate (HR) and PGA may also be used to detect inconsistent behavior of PGA.

FIGS. 6a to 6d illustrate measurements made in a hospital environment in an abnormal situation where inconsistent PGA behavior has been detected during a surgery. The measurements are made according to the embodiment of FIG. 5. FIG. 6a illustrates measured logPGA values, FIG. 6b illustrates an index of nociception calculated based on PG and ECG signals, FIG. 6c illustrates measured PTT values, and FIG. 6d illustrates the quality measure calculated as the correlation of the logPGA and PTT values. FIG. 6a further shows the moment of incision. During the incision the index of nociception behaves inconsistently, but during the rest of the surgery again consistently. The quality measure shown in FIG. 6d reveals the inconsistent behavior of the index. In this case, the scale of the quality measure may be interpreted approximately as follows:
1 . . . 0.6: patient response is consistent, high quality
0.6 . . . −0.6: no significant inconsistencies, medium quality
−0.6 . . . −1: patient response is probably not consistent, low quality.

In some embodiments of the invention, the apparatus of the invention monitors the physiological signals and/or the parameters and attempts to keep the quality of the index as high as possible based on the monitoring process For this purpose, the apparatus may automatically select the physiological signal(s) to be used for the determination of the index from among the physiological signals available and update the selection in the course of the index determination, if necessary. For example, if a new physiological signal that has relevant information content becomes available, the apparatus may add the signal to the set of signals used for the determination of the index.

Each update in the physiological signals used for the index determination is accompanied by a corresponding change in the algorithm used for the calculation of the index. The algorithm to be used for the calculation of the index is thus selected based on the particular physiological signals employed at each time. Quality control information indicating the selected physiological signal(s) and the related algorithm may thus be produced based on the information about the physiological signal(s) available. The said information may further be coded to visual cues in different ways. As discussed above, one possibility here is to map each signal combination to respective reliability level and to indicate the current reliability level to the user. However, the quality control information may also be utilized to enhance the reliability of the index without giving a visual indication of the reliability.

Similarly to the above addition of a physiological signal, the apparatus may also remove one of the physiological signals used for the determination of the index, if the quality/reliability of the measurement drops below a certain limit or if the deterioration of the quality/reliability can be identified to be due to a particular signal.

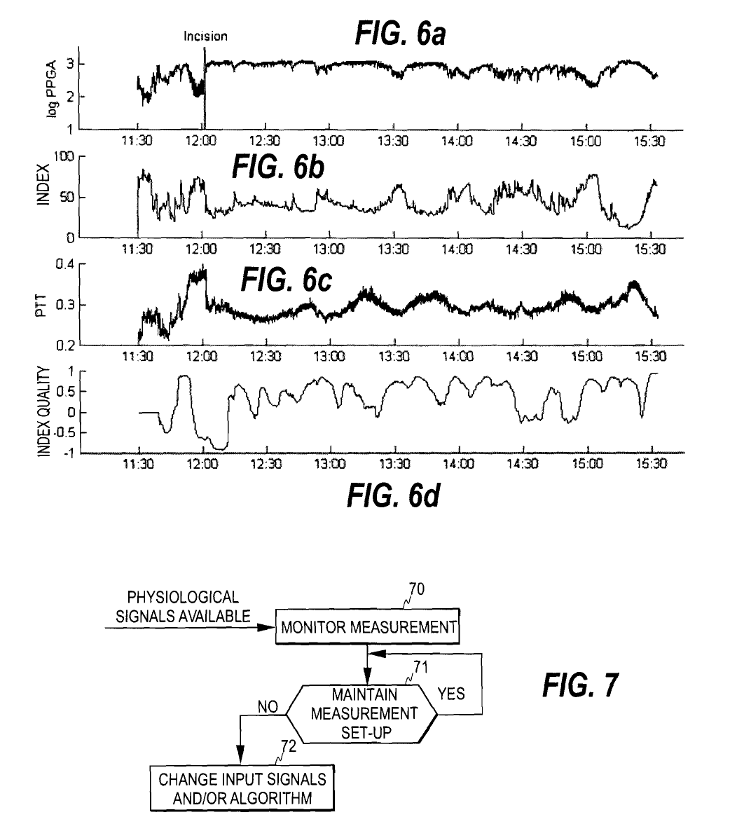

FIG. 7 illustrates an example of the above embodiments of the invention. In this example, the measurement, i.e. the process of the determination of the index, is continuously monitored at step 70.

If an event is detected at step 71 that indicates a need to improve the reliability of the index, the process changes the set of signals/parameters and/or the calculation algorithm used for the determination of the index.

If, for example, a change in the physiological signals available or the quality information indicates that the quality drops below a certain limit, and if additional physiological signals are not available at the moment, i.e. if it is not possible to add or replace a physiological signal, the algorithm may be changed. This may be carried out by changing the relative weights of the parameter sequences, for example. For example, if it is detected in the embodiments of FIG. 2 or FIG. 4 that the quality drops below a certain limit since the PGA data does not behave consistently with PTT or (NI)BP data, the process may use the ECG signal for the determination of the diagnostic index for the duration of the period of inconsistency. When dropping the PG signal from the index determination, the process may also add the number of parameters, i.e. parameter sequences, derived from the ECG signal. For example, from the ECG signal the process may derive a first time series representing normalized pulse intervals and a second time series representing the (normalized) variability of the pulse intervals.

An event detected at step 71 may also indicate that a new physiological signal has become available. If this signal has relevant information content with respect to the determination of the index, quality control information may be produced, which indicates that the said physiological signal is to be added to the set of signals used for the determination of the index. The algorithm used for the determination is updated accordingly. If, for example, invasive blood pressure is taken into use during surgery, it may be introduced to the set of physiological signals based on which the diagnostic index is determined by adding it to the said set or by replacing one of the signals in the set, such as NIBP. The quality control information indicating the signal(s)/parameter(s) and the relevant algorithm to be used for the determination of the index may be produced continuously or only when a change is made in the measurement set-up.

A change of the index structure, for instance the replacement of a bad quality physiological signal or parameter sequence by a new physiological signal or parameter sequence may be carried out easily: the new parameter sequence is first normalized to scale the output parameter sequence to a predetermined range, such as 0 to 100. Because the bad old sequence was similarly normalized, the new sequence can replace the old one by maintaining the same weighting factor. So, the new parameter sequence simply replaces the old one. In case a new index component to the diagnostic index is taken into use, the new weight factors may be calculated, after the normalization of all parameter sequences, as proportional to their respective quality index estimates. For example, the old index may be structured as to contain 70% of normalized PGA and 30% of normalized HR information. If now the quality of the PGA information is deteriorated, but still the HR quality is maintained as perfect, the new index may be calculated as 30% of normalized HR, 30% of normalized PGA, and 40% of a normalized new parameter sequence, say for instance BP or PTT. According to an embodiment of a dynamically adjusted diagnostic index structure all the input physiological parameter sequences are thus normalized as usual and the output index structure is determined based on the respective signal qualities. If two signal qualities are perfect, i.e. 100% quality, and a third one is less than 100%, the diagnostic index may be calculated from the two best quality signals. If all signal qualities are less than 100%, new weighting factors (Wᵢ) may be calculated, which are proportional to their quality ratings (Qᵢ) and their nominal weighting factors (NWᵢ) assuming perfect signals. An optimization routine may be based on an optimizing algorithm, in which the overall quality Q=Σ(Qᵢ*NWᵢ) is maximized under constriction that the new weighting factors, Wᵢ=Qᵢ*NWᵢ, sum up, after proper scaling, to one.

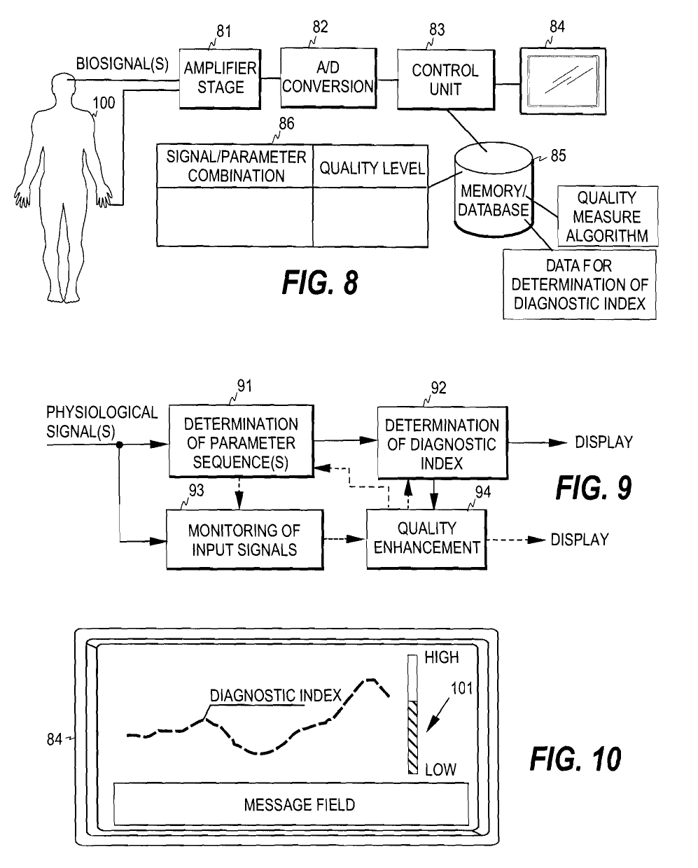

FIG. 8 illustrates one embodiment of the system or apparatus according to the invention. The physiological signal(s) obtained from one or more sensors attached to a patient 100 are supplied to an amplifier stage 81, which amplifies the signal(s) before they are sampled and converted into digitized format in an A/D converter 82. The digitized signals are supplied to a control unit 83 which may comprise one or more processors.

The control unit is provided with a memory or database 85 holding the digitized signal data obtained from the sensor(s). The control unit may produce the time series needed and determine the diagnostic index based on the time series. For this purpose, the memory may store the algorithms and parameters needed for the determination of the diagnostic index. Furthermore, the memory may store the algorithm(s)/rule(s) needed for generating quality information indicative of the reliability of the diagnostic index. These may include tables 86 which map a certain signal or parameter combination to a quality level or an algorithm for determining a quality measure, such as the measure indicative of the correlation between two parameters. As shown in FIG. 9, the control unit may include four operational modules or units: a first calculation unit 91 configured to derive the parameter sequence(s), a second calculation unit 92 configured to form the diagnostic index based on the parameter sequence(s), a monitoring unit 93 configured to monitor the physiological signals and/or the parameter sequence(s), and a quality enhancement module 94 configured to enhance the quality of the diagnostic index. As discussed above, in some embodiments the module may include a calculation unit for deriving data indicative of the current reliability of the index and a display driver configured to control a display/monitor 84. In some embodiments, the module may control the determination of the index to enhance the reliability of the index. Depending on the available signals/parameters, the quality enhancement module 94 may warn the user of various situations in which the reliability of the measurement may be compromised. The following table shows some additional abnormal situations, the corresponding input signals/parameters that may be monitored in the monitoring unit to detect a particular situation, and the event that indicates lowered reliability.

| ABNORMAL | MONITORED | Event indicating |
|---|---|---|
| SITUATION | SIGNAL/PARAMETER | lowered reliability |
| hypovolemia | Systolic pressure variability | SPV or PGA |
| | (SPV), PGA variability | variability increases |
| | | above a certain |
| | | threshold value |
| hypothermia | PGA, Perfusion Index (PI), | PI smaller than a |
| | Peripheral or core temperature, | certain threshold |
| | Difference of the core and | value, the peripheral |
| | peripheral temperature | temperature or the |
| | | difference to the core |
| | | temperature beyond |
| | | a set threshold |
| patient awake | Entropy, Bispectral Index (BIS), | Entropy/BIS larger |
| | motion signal from a pulse | than a set threshold. |
| | oximeter | Frequent movements |
| | | detected. |
| strong | BP, HR, PGA | After increase of BP, |
| baroreflex | | HR decreases and |
| | | PGA increases with |
| | | a typical baroreflex |
| | | pattern. |
| neuropathy | HRV, PGA variability | HRV and PGA |
| | | variability smaller |
| | | than a set threshold |
| arrhythmia | HRV, Pulse rate variability, | Arrhythmia detected |
| | parameter alarms | from ECG or BP or |
| | | PG |
| external/ | noise | Poor PG, ECG |
| internal device | | |

Although one computer unit or processor may perform the above steps of the control unit, the processing of the data may also be distributed among different units/processors (servers) within a network, such as a hospital LAN (local area network). The apparatus of the invention may thus also be implemented as a distributed system.

The control unit may display the results through at least one monitor 84 connected to the control unit. By showing the reliability level of the diagnostic index on the screen of the monitor, the apparatus acts as decision-support tool for the physician, such as an anesthesiologist. FIG. 10 illustrates an example of the indication of the diagnostic index and the reliability level thereof. The diagnostic index, shown as a dashed line, may be displayed as a graph that indicates the trend of the index. The reliability level of the current value of the diagnostic index may be shown as a bar 101 whose height increases as the reliability increases.

A measurement device determining the diagnostic index may also be upgraded to improve the quality of the diagnostic index. Such an upgrade may be implemented by delivering to the measurement device a software module that enables the device to monitor the measurement and to indicate the quality of the index in one of the above-described manners. The software module may be delivered, for example, on a data carrier, such as a CD or a memory card, or through a telecommunications network. The software module is provided with access to memory so that it can retrieve the data necessary for determining the reliability level. The content of the software module depends on the measurement device; if the measurement device is capable of determining the diagnostic index, the software module includes only a first portion configured to monitor the measurement and a second portion configured to enhance the quality of the measurement in response to the information obtained by the first portion. If the measurement device lacks the ability to determine the index but stores data from which the index may be derived, the software module further includes a portion configured to form the diagnostic index based on the said data.

Although the invention was described above with reference to the examples shown in the appended drawings, it is obvious that the invention is not limited to these, but may be modified by those skilled in the art without departing from the scope and spirit of the invention.

We claim:

1. A method for ascertaining the clinical state of a subject, the method comprising:
acquiring at least one physiological signal from a subject;
deriving at least one parameter sequence from at least one desired physiological signal belonging to the at least one physiological signal;
performing an index determination process, thereby to form a diagnostic index based on the at least one parameter sequence, wherein the diagnostic index serves as a measure of the clinical state of the subject and wherein the index determination process includes indicating the diagnostic index to a user;
monitoring at least one input signal, wherein the at least one input signal belongs to a group including the at least one physiological signal and the at least one parameter sequence; and
enhancing, based on the monitoring, the quality of the index determination process.

2. A method according to claim 1, wherein the enhancing includes:
producing, based on the at least one input signal, quality information indicative of the reliability of the diagnostic index; and
giving, based on the quality information, an indication of the current reliability of the diagnostic index.

3. A method according to claim 2, wherein the monitoring comprises:
determining changes occurring in a selected physiological signal belonging to the at least one physiological signal; and
defining changes occurring in a selected parameter sequence belonging to the at least one parameter sequence; and
the producing comprises
comparing the changes occurring in the selected physiological signal with the changes occurring in the selected parameter sequence; and
generating the quality information based on the comparing.

4. A method according to claim 3, wherein the determining includes determining changes occurring in the selected physiological signal, wherein the selected physiological signal is a blood pressure (BP) signal.

5. A method according to claim 3, wherein the defining includes defining changes occurring in the selected parameter sequence, wherein the selected parameter sequence is a plethysmographic amplitude (PGA) signal.

6. A method according to claim 2, wherein:
the producing includes calculating the quality information as the correlation between a selected physiological signal and a selected parameter sequence, wherein the selected physiological signal belongs to the at least one physiological signal and the selected parameter sequence belongs to the at least one parameter sequence; and
the giving includes displaying a warning message when the correlation fulfills a predetermined condition.

7. A method according to claim 2, wherein:
the monitoring includes determining an auxiliary signal based on at least one of the at least one physiological signal; and
the producing includes producing the quality information, in which the quality information represents the correlation between the auxiliary signal and a selected parameter sequence belonging to the at least one parameter sequence.

8. A method according to claim 7, wherein the producing includes producing the correlation between the auxiliary signal and the selected parameter sequence, in which the selected parameter sequence is a plethysmographic amplitude (PGA) signal.

9. A method according to claim 7, wherein the determining includes determining the auxiliary signal, in which the auxiliary signal is one of a signal representing the pulse transit time (PTT) of the subject and a signal representing the heart rate of the subject.

10. A method according to claim 7, wherein the giving includes displaying a warning message when the correlation fulfills a predetermined condition.

11. A method according to claim 2, wherein the producing includes producing the quality information, in which the quality information is a quality measure indicative of the current reliability of the diagnostic index.

12. A method according to claim 2, wherein the enhancing includes changing at least one of the at least one parameter sequence if the quality information indicates that the current reliability of the diagnostic index drops below a certain limit.

13. A method according to claim 2, wherein the enhancing includes transforming the quality information into visually interpretable symbols.

14. A method according to claim 1, wherein:
the monitoring includes monitoring the at least one physiological signal acquired from the subject; and
the enhancing includes generating an indication of the type(s) of the at least one physiological signal.

15. A method according to claim 14, wherein the enhancing further includes:
mapping the indication to a quality level; and
indicating the quality level visually to a user.

16. A method according to claim 14, wherein the enhancing further includes selecting, based on the monitoring, the at least one desired physiological signal from among the at least one physiological signal and an algorithm for the index determination process.

17. A method according to claim 16, further comprising repeating the selecting during the performing of the index determination process.

18. A method according to claim 17, wherein the repeating is performed in response to a change in the number of the at least one physiological signal.

19. An apparatus for ascertaining the clinical state of a subject, the apparatus comprising:
a measurement unit configured to acquire at least one physiological signal from a subject;
a first calculation unit configured to derive at least one parameter sequence from at least one desired physiological signal belonging to the at least one physiological signal;
a second calculation unit configured to perform an index determination process, thereby to form a diagnostic index based on the at least one parameter sequence, wherein the diagnostic index serves as a measure of the clinical state of the subject and wherein the index determination process is configured to indicate the diagnostic index to a user;
a quality enhancing module configured to monitor at least one input signal and to enhance the quality of the index determination process based on the at least one input signal, wherein the at least one input signal belongs to a group including the at least one physiological signal and the at least one parameter sequence.

20. An apparatus according to claim 19, wherein the quality enhancing module is configured to:
produce quality information indicative of the reliability of the diagnostic index; and
give, based on the quality information, an indication of the current reliability of the diagnostic index.

21. An apparatus according to claim 20, wherein the quality enhancing module is configured to:
determine changes occurring in a selected physiological signal, wherein the selected physiological signal belongs to the at least one physiological signal;
define changes occurring in a selected parameter sequence, wherein the selected parameter sequence belongs to the at least one parameter sequence; and
compare the changes occurring in the selected physiological signal with the changes occurring in the selected parameter sequence.

22. An apparatus according to claim 21, wherein the selected physiological signal is a blood pressure (BP) signal.

23. An apparatus according to claim 21, wherein the selected parameter sequence is a plethysmographic amplitude (PGA) signal.

24. An apparatus according to claim 20, wherein the quality enhancing module is configured to calculate a correlation between a selected physiological signal and a selected parameter sequence, wherein the selected physiological signal belongs to the at least one physiological signal and the selected parameter sequence belongs to the at least one parameter sequence.

25. An apparatus according to claim 20, wherein the quality enhancing module is configured to:
determine an auxiliary signal based on at least one of the at least one physiological signal;
determine changes occurring in the auxiliary signal;
define changes occurring in a selected parameter sequence, wherein the selected parameter sequence belongs to the at least one parameter sequence; and
compare the changes occurring in the auxiliary signal with the changes occurring in the selected parameter sequence.

26. An apparatus according to claim 25, wherein the selected parameter sequence is a plethysmographic amplitude (PGA) signal and the auxiliary signal is one of a signal representing the pulse transit time (PTT) of the subject and a signal representing the heart rate of the subject.

27. An apparatus according to claim 26, wherein the quality enhancing module is configured to calculate a correlation between the auxiliary signal and the selected parameter sequence.

28. An apparatus according to claim 20, wherein the quality enhancing module is configured to determine a quality measure indicative of the current reliability of the diagnostic index.

29. An apparatus according to claim 20, wherein the quality enhancing module is configured to change at least one of the at least one parameter sequence if the quality information indicates that the current reliability of the diagnostic index drops below a certain limit.

30. An apparatus according to claim 20, wherein the quality enhancing module is configured to transform the quality information into visually interpretable symbols.

31. An apparatus according to claim 19, wherein quality enhancing module is configured to:
monitor the at least one physiological signal acquired from the subject; and
generate an indication of the types of the at least one physiological signal.

32. An apparatus according to claim 31, wherein the quality enhancing module is further configured to map the indication to a quality level and to display the quality level visually to a user.

33. An apparatus according to claim 31, wherein the quality enhancing module is further configured to select the at least one desired physiological signal and an algorithm for the second calculation unit.

34. An apparatus according to claim 31, wherein the quality enhancing module is further configured to select the at least one desired physiological signal in response to a change in the number of the at least one physiological signal.

35. An apparatus for ascertaining the clinical state of a subject, the apparatus comprising:
measurement means for acquiring at least one physiological signal from a subject;
first calculation means for deriving at least one parameter sequence from at least one desired physiological signal belonging to the at least one physiological signal;
second calculation means for performing an index determination process configured to form a diagnostic index based on the at least one parameter sequence, wherein the diagnostic index serves as a measure of the clinical state of the subject and wherein the index determination process is configured to indicate the diagnostic index to a user;
quality enhancing means for monitoring at least one input signal and for enhancing the quality of the index determination process based on the at least one input signal, wherein the at least one input signal belongs to a group including the at least one physiological signal and the at least one parameter sequence.

36. A computer program product for improving the quality of a measurement of the clinical state of a subject, the computer program product comprising:
a first program code portion configured to monitor at least one input signal belonging to a group including at least one physiological signal acquired from a subject and at least one parameter sequence derived from at least one desired physiological signal belonging to the at least one physiological signal; and
a second program code portion responsive to the first program code portion and configured to enhance the quality of an index determination process configured to determine a diagnostic index based on the at least one parameter sequence, wherein the diagnostic index serves as a measure of the clinical state of the subject.

37. A computer program product according to claim 36, wherein the computer product further comprises a third program code portion configured to perform the index determination process.
